(19) United States
(12) Patent Application Publication
(10) Pub. No.: US 2004/0247647 A1
(43) Pub. Date: Dec. 9, 2004

(54) Rapidly dissolving edible film compositions with cellulose film forming polymers

(75) Inventors: Alisa Ann Ivory, Cincinnati, OH (US); James Milton Rossman, Tampa, FL (US); Kuo-Chung Mark Lee, Hamilton, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/810,939

(22) Filed: Mar. 26, 2004

Related U.S. Application Data
(60) Provisional application No. 60/457,714, filed on Mar. 26, 2003.

(51) Int. Cl. A61K 9/68

(52) U.S. Cl. 424/440

(57) ABSTRACT

The present invention relates to an edible film composition comprising: a safe and effective amount of a cellulose based film forming agent comprising a mixture of at least one low viscosity cellulose based film forming agent and at least one high viscosity cellulose based film forming agent; a safe and effective amount of a plasticizing agent; and a safe and effective amount of a flavoring agent; wherein the film composition rapidly dissolves in the oral cavity. This invention further relates to a method of increasing film strength of an edible film composition while maintaining rapid film dissolution, by incorporating the above described cellulose based film forming agents into an edible film composition. In one embodiment the edible film is a breath freshening film.

### CROSS-REFERENCE TO RELATED APPLICATION

This application claims the benefit of U.S. Provisional Application No. 60/457,714, filed Mar. 26, 2003.

### FIELD OF THE INVENTION

The present invention relates to an edible film composition comprising a combination of a high viscosity film forming agent and a low viscosity film forming agent for delivering breath freshening ingredients, oral care active ingredients, and/or pharmaceutical active ingredients to the oral cavity. The edible film composition has improved film strength, relatively low levels of the film former, while maintaining complete and rapid film dissolution.

### BACKGROUND OF THE INVENTION

Oral malodor, plaque, gingivitis, caries, periodontal disease and other oral care conditions are conditions that effect many people. For example, oral malodor, also known as halitosis or bad breath has been estimated to afflict about 50-90 million people in the United States. To combat the above oral care diseases or conditions, a variety of products have been developed including oral rinses, dentifrices, toothgels, chewing gums, lozenges and mints, etc. The use of these products, especially chewing gum and confectioneries, is not always convenient or socially acceptable as they require a brushing, rinsing, sucking or chewing action on the part of the consumer over an extended period of time which can be inconvenient, time consuming, or distracting in a social or business setting.

The prior art teaches edible, consumable films adapted to dissolve in the oral cavity containing flavoring agents or other breath freshening agents. For example, WO 00/18365, Warner-Lambert, published Apr. 6, 2000, teaches a breath freshening film adapted to dissolve in the mouth of a consumer comprised of a water soluble polymer such as pullulan or hydroxypropylmethyl cellulose and an essential oil selected from thymol, methyl salicylate, eucalyptol and/or menthol. In addition U.S. Pat. No. 5,948,430, issued Sep. 7, 1999, assigned to LTS Lohmann, discloses a film composition containing therapeutic and/or breath freshening agents, prepared from water soluble polymers such as hydroxypropylmethyl cellulose, hydroxypropylcellulose, etc., and a polyalcohol. This reference alleges that these films, when applied to the oral cavity, exhibit instant wettability followed by rapid dissolution. Furthermore, U.S. Pat. No. 6,419,903, issued Jul. 16, 2002, assigned to Colgate, teaches consumable films that comprise hydroxyalkylmethylcellulose as a film forming agent, pre-gelatinezed starch, and a flavoring agent.

Despite the above noted disclosures of dissolvable, edible films, there is still a need for improvement in such films, namely increasing the film strength to avoid breakage or curling of the film during storage or upon processing (e.g. casting, cutting and/or packing). Also, increasing the film strength avoids breakage of the film upon consumer dispensing of the film for use. The present invention provides increased film strength while maintaining complete and rapid dissolution of the film in the oral cavity. Rapid and complete dissolution of the edible film when placed in the oral cavity, is advantageous since the undissolved film residue imparts an unacceptable, unpalatable, slimy feel to the palate of the user.

### SUMMARY OF THE INVENTION

The present invention relates to an edible film composition comprising: a safe and effective amount of a mixture of at least one low viscosity cellulose film forming agent and at least one high viscosity cellulose film forming agent; a safe and effective amount of a plasticizing agent; and a safe and effective amount of a flavoring agent; wherein the film composition completely and rapidly dissolves in the oral cavity. This invention further relates to a method of increasing the film strength of an edible film composition while maintaining complete and rapid film dissolution, by incorporating the above mixture of at least one low viscosity cellulose film forming agent and at least one high viscosity cellulose film forming agent into the edible film composition. The invention further relates to a method of treating or preventing an oral and/or respiratory condition by administering a safe and effective amount of the above composition to the oral cavity of a subject in need thereof. In one embodiment the edible film is a breath freshening film.

### DETAILED DESCRIPTION OF THE INVENTION

Definitions

By “anticalculus” or “antitartar” agent, as used herein, means a material effective in reducing, controlling, inhibiting, preventing, and/or minimizing mineral (e.g., calcium phosphate) deposition related to calculus or tartar formation.

By “safe and effective amount” as used herein is meant an amount of a component, high enough to significantly (positively) modify the condition to be treated or to effect the desired result, but low enough to avoid serious side effects (at a reasonable benefit/risk ratio), within the scope of sound medical/dental judgment. The safe and effective amount of a component, will vary with the particular condition (e.g., to control breath malodor) being treated, the age and physical condition of the patient being treated, the severity of the condition, the duration of treatment, the nature of concurrent therapy, the specific form employed, and the particular vehicle from which the component is applied.

By “rapidly dissolves” or “rapid dissolution” as used herein is meant that the edible film dissolves in about 1 seconds to about 60 seconds, in another embodiment dissolves in about 3 seconds to about 50 seconds, in another embodiment dissolves in about 4 seconds to about 40 seconds, in another embodiment dissolves in about 5 seconds to about 20 seconds, once the subject places the film in the oral cavity.

All percentages and ratios used hereinafter are by weight of total composition, unless otherwise indicated. As used herein, percentage by weight of the film composition means percent by weight of the wet film composition, unless otherwise indicated.

All measurements referred to herein are made at 25° C. unless otherwise specified.

All percentages, ratios, and levels of ingredients referred to herein are based on the actual amount of the ingredient, and do not include solvents, fillers, or other materials with which the ingredient may be combined as a commercially available product, unless otherwise indicated.

All publications, patent applications, and issued patents mentioned herein are hereby incorporated in their entirety by reference. Citation of any reference is not an admission regarding any determination as to its availability as prior art to the claimed invention.

Herein, “comprising” means the term “comprising” and can include “consisting of” and “consisting essentially of.”

### Cellulose Based Film Forming Agent

The compositions of the present invention comprise a safe and effective amount of a mixture of cellulose based film forming agents. In particular the film forming agent of the present invention comprises a mixture of at least one low viscosity cellulose based film forming agent and at least one high viscosity cellulose based film forming agent.

The low viscosity film forming agents used herein have a viscosity from about 1 to about 40 millipascal seconds (mPa.s), in another embodiment from about 2 to about 20 mPa.s, in another embodiment from about 2 to about 4 mPa.s. The high viscosity film forming agents used herein have a viscosity from about 50 to about 10,000 millipascal seconds (mPa.s), in another embodiment from about 70 to about 1,000 mPa.s, in another embodiment from about 100 to about 5,000 mpa.s. These viscosities are determined as a 2% by weight aqueous solution of the film forming agent at 20 degrees C. using a Ubbelohde tube viscometer.

The cellulose based film forming agents are selected from the group consisting of methyl cellulose, carboxymethylcellulose, hydroxyethylcellulose, hydroxypropylcellulose, hydroxy-propylmethylcellulose, and mixtures thereof, in another embodiment is selected from the group consisting of hydroxypropylcellulose, hydroxy-propylmethylcellulose, and mixtures thereof, in yet another embodiment is hydroxypropylmethylcellulose (HPMC). Particularly preferred HPMCs are available commercially from the Dow Chemical Company under the trade designation of Methocel K4M (viscosity of 4,000 mPa.s); Methocel K 100 (viscosity of 100 mPa.s); Methocel K3 (viscosity of 3 mPa.s); Methocel E 50 (viscosity of 50 mPa.s); Methocel E4M (viscosity of 4,000 mPa.s). The Methocel K series has a 19-24% methoxy group substitution and a 7-12% hydroxyproproxyl group substitution. The Methocel E series has a 28-30% methoxy group substitution and a 7-12% hydroxyproproxyl group substitution.

In one embodiment either the low viscosity cellulose based film forming agent and/or the high viscosity cellulose based film forming agent is HPMC with a 19-24% methoxy group substitution and a 7-12% hydroxyproproxyl group substitution.

In general lower levels (thereby reducing costs) of the film forming agent can be used herein. The present compositions comprise, in one embodiment, from about 2% to about 30%, in another embodiment from about 3% to about 20%, in yet another embodiment from about 4% to about 7%, by weight of the wet composition, of total film forming agent(s). In one embodiment the level of the low viscosity cellulose based film forming agent is from about 0.1 to about 3%, in another embodiment from about 0.5% to about 2%, by weight of the wet composition.

Using the mixture of film forming agent as described herein, provides good film strength while maintaining rapid film dissolution, while also minimizing “curling” of the film during cutting and packing of the film into a container for end use by the consumer. This is achieved despite relatively low levels of film forming agent.

In addition to the above essential film forming agents, the present composition may also comprise additional film forming agents. In this regard any water soluble or water dispersible film forming agent can be used herein. In one embodiment the additional film forming agent is selected from the group consisting of polyvinyl pyrrolidone, polyvinyl alcohol, sodium alginate, polyethylene glycol, natural gums, xanthan gum, tragacanth gum, guar gum, acacia gum, arabic gum, polyacrylic acid, methylmethacrylate copolymer, carboxyvinyl polymer, polyvinyl pyrrolidone, amylose, high amylose starch, hydroxypropylated high amylose starch, pullulan, dextrin, pectin, chitin, chitosan, levan, elsinan, collagen, gelatin, zein, gluten, soy protein isolate, whey protein isolate, casein, and mixtures thereof.

### Plasticizing Agent

The compositions of the present invention also comprise a safe and effective amount of a plasticizing agent to improve flexibility and reduce brittleness of the edible film composition. In one embodiment the level of the plasticizing agent ranges from about 0.01% to about 30%, in another embodiment from about 1% to about 10%, in another embodiment from about 2% to about 5%, by weight of the dry film composition.

Suitable plasticizing agents of the present invention include, but are not limited to, polyols (such as sorbitol; glycerin; polyethylene glycol; propylene glycol; acetylated monoglyceride; hydrogenated starch hydrolysates; corn syrups; and derivatives thereof; xylitol; glycerol monoesters with fatty acids; triacetin; diacetin; and monoacetin; and mixtures thereof. In one embodiment the plasticizing agent of the present invention is propylene glycol.

### Flavoring Agent

The compositions of the present invention also comprise a safe and effective amount of a flavoring agent. Suitable flavoring agents include oil of wintergreen, oil of peppermint, oil of spearmint, clove bud oil, menthol, anethole, methyl salicylate, eucalyptol, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.1% to about 60%, in another embodiment from about 15% to about 40%, in yet another embodiment from about 25% to about 35%, by weight of the dry film composition. In another embodiment the flavors are used at much higher levels in order to provide greater flavor impact for example are present at a level of from about 10 wt % to about 35% wt in another embodiment from about 15 wt % to about 30 wt %, in another embodiment from about 18 wt % to about 25 wt %, of the dry film composition.

In another embodiment, in order to stabilize the flavor, the compositions optionally comprise a vegetable oil selected from the group consisting of corn, soybean, cottonseed, linseed, olive, peanut, castor, palm and coconut oils, in yet another embodiment the vegetable oil is canola oil.

Vegetable oils are generally used in the compositions at levels of from about 0.1% to about 20%, in another embodiment from about 1% to about 5%, in yet another embodiment from about 2% to about 4%, by weight of the dry film composition.

### Optional Active Agents

The present invention may optionally comprise a safe and effective amount of an oral care active agent and/or a pharmaceutical active agent. The oral care and pharmaceutical active agents are described in detail hereinbelow.

Oral Care Active Agent

The oral care active agent suitable for use herein is selected from the group consisting of anticalculus agent, fluoride ion source, antimicrobial agents, dentinal desensitizing agents, anesthetic agents, antifungal agents, anti-inflammatory agents, selective H-2 antagonists, anticaries agents, nutrients, and mixtures thereof. The oral care active agent preferably contains an active at a level where upon directed use, the benefit sought by the wearer is promoted without detriment to the oral surface to which it is applied. Examples of the “oral conditions” these actives address include, but, are not limited to, appearance and structural changes to teeth, whitening, stain removal, plaque removal, tartar removal, cavity prevention and treatment, inflamed and/or bleeding gums, mucosal wounds, lesions, ulcers, aphthous ulcers, cold sores, tooth abscesses, and the elimination of mouth malodor resulting from the conditions above and other causes such as microbial proliferation.

Suitable oral care actives include any material that is generally considered safe for use in the oral cavity and that provides changes to the overall appearance and/or health of the oral cavity. The level of oral care substance in the compositions of the present invention is generally, unless specifically noted, from about 0.01% to about 50%, preferably from about 0.1% to about 20%, more preferably from about 0.5% to about 10%, and even more preferably from about 1% to about 7%, by weight of the dry film composition.

### Anticaries Agents and Fluoride Ion Source

The present composition may comprise a safe and effective amount of an anticaries agent, and mixtures thereof. In one embodiment the anticaries agent is selected from the group consisting of xylitol, fluoride ion source, and mixtures thereof. The fluoride ion source provides free fluoride ion during the use of the composition. In one embodiment the oral care active agent is a fluoride ion source selected from the group consisting of sodium fluoride, stannous fluoride, indium fluoride, organic fluorides such as amine fluorides and sodium monofluorophosphate. Sodium fluoride is the fluoride ion in another embodiment. Norris et al., U.S. Pat. No. 3,678,154 issued Jul. 1, 1972, discloses such fluoride salts as well as others that can be used as the fluoride ion source.

The present composition may optionally contain a safe and effective amount of a fluoride ion source in another embodiment the level is from about 50 ppm to about 3500 ppm, in another embodiment from about 100 ppm to about 30,000 ppm, and in another embodiment from about 200 ppm to about 2,800 ppm, and in another embodiment from about 500 ppm to about 1,500 ppm, of free fluoride ions.

Anticalculus Agents

The present compositions may comprise a safe and effective amount of at least one anticalculus agent. This amount is generally from about 0.01% to about 40% by weight of the composition, in another embodiment is from about 0.1% to about 25%, and in yet another embodiment is from about 4.5% to about 20%, and in yet another embodiment is from about 5% to about 15%, by weight of the composition. The anticalculus agent should also be essentially compatible with the other components of the composition.

In one embodiment the anticalculus agent is selected from the group consisting of polyphosphates and salts thereof; diphosphonates and salts thereof; and mixtures thereof. In another embodiment the anticalculus agent is selected from the group consisting of pyrophosphate, polyphosphate, and mixtures thereof.

Polyphosphate

In one embodiment of the present invention, the anticalculus agent is a polyphosphate. A polyphosphate is generally understood to consist of two or more phosphate molecules arranged primarily in a linear configuration, although some cyclic derivatives may be present. Linear polyphosphates correspond to (X PO₃)ₙ where n is about 2 to about 125, wherein preferably n is greater than 4, and X is for example sodium, potassium, etc. For (X PO₃)ₙ when n is at least 3 the polyphosphates are glassy in character. Counterions for these phosphates may be the alkali metal, alkaline earth metal, ammonium, C₂-C₆ alkanolammonium and salt mixtures. Polyphosphates are generally employed as their wholly or partially neutralized water soluble alkali metal salts such as potassium, sodium, ammonium salts, and mixtures thereof. The inorganic polyphosphate salts include alkali metal (e.g. sodium) tripolyphosphate, tetrapolyphosphate, dialkyl metal (e.g. disodium) diacid, trialkyl metal (e.g. trisodium) monoacid, potassium hydrogen phosphate, sodium hydrogen phosphate, and alkali metal (e.g. sodium) hexametaphosphate, and mixtures thereof. Polyphosphates larger than tetrapolyphosphate usually occur as amorphous glassy materials. In one embodiment the polyphosphates are those manufactured by FMC Corporation which are commercially known as Sodaphos (n≈6), Hexaphos (n≈13), and Glass H (n≈21), and mixtures thereof. The present compositions will typically comprise from about 0.5% to about 20%, in one embodiment from about 4% to about 15%, in yet another embodiment from about 6% to about 12%, by weight of the composition of polyphosphate.

The phosphate sources are described in more detail in Kirk & Othmer, Encyclopedia of Chemical Technology, Fourth Edition, Volume 18, Wiley-Interscience Publishers (1996), pages 685-707, incorporated herein by reference in its entirety, including all references incorporated into Kirk & Othmer.

In one embodiment the polyphosphates are the linear “glassy” polyposphates having the formula:

XO(XPO₃)ₙX

wherein X is sodium or potassium; and n averages from about 6 to about 125.

In one embodiment, when n is at least 2 in either of the above polyphosphate formulas, the level of anticalculus agent is from about 4.5% to about 40%, in another embodiment is from about 5% to about 25%, and in even another embodiment is from about 8% to about 15%, by weight of the composition. Polyphosphates are disclosed in U.S. Pat. No. 4,913,895.

Pyrophosphate

The pyrophosphate salts useful in the present compositions include, alkali metal pyrophosphates, di-, tri-, and mono-potassium or sodium pyrophosphates, dialkali metal pyrophosphate salts, tetraalkali metal pyrophosphate salts, and mixtures thereof. In one embodiment the pyrophosphate salt is selected from the group consisting of trisodium pyrophosphate, disodium dihydrogen pyrophosphate (Na₂H₂P₂O₇), dipotassium pyrophosphate, tetrasodium pyrophosphate (Na₄P₂O₇), tetrapotassium pyrophosphate (K₄P₂O₇), and mixtures thereof. The pyrophosphate salts described in U.S. Pat. No. 4,515,772, issued May 7, 1985, and U.S. Pat. No. 4,885,155, issued Dec. 5, 1989, both to Parran et al., are incorporated herein by reference in their entirety, as well as the references disclosed therein. The pyrophosphate salts are described in more detail in Kirk & Othmer, Encyclopedia of Chemical Technology, Third Edition, Volume 17, Wiley-Interscience Publishers (1982), pages 685-707, incorporated herein by reference in its entirety, including all references incorporated into Kirk & Othmer.

In one embodiment, the compositions of the present invention comprise tetrasodium pyrophosphate. Tetrasodium pyrophosphate may be the anhydrous salt form or the decahydrate form, or any other species stable in solid form in the present compositions. The salt is in its solid particle form, which may be its crystalline and/or amorphous state, with the particle size of the salt preferably being small enough to be aesthetically acceptable and readily soluble during use.

The level of pyrophosphate salt in the compositions of the present invention is any safe and effective amount, and is generally from about 1.5% to about 15%, in another embodiment from about 2% to about 10%, and yet in another embodiment from about 3% to about 8%, by weight of the composition.

The level of pyrophosphate salt in the compositions of the present invention is any safe and effective amount, and is generally from about 1.5% to about 15%, in another embodiment from about 2% to about 10%, and yet in another embodiment from about 3% to about 8%, by weight of the composition.

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., the disclosure of which is incorporated herein by reference in its entirety; as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

Antimicrobial Agents and Antifungal Agents

Antimicrobial antiplaque agents may also by optionally present in the present compositions. Such agents may include, but are not limited to, triclosan, 5-chloro-2-(2,4-dichlorophenoxy)-phenol, as described in The Merck Index, 11th ed. (1989), pp. 1529 (entry no. 9573) in U.S. Pat. No. 3,506,720, and in European Patent Application No. 0,251,591 of Beecham Group, PLC, published Jan. 7, 1988; chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; effective antimicrobial amounts of essential oils and combinations thereof for example citral, geranial, and combinations of menthol, eucalyptol, thymol and methyl salicylate; antimicrobial metals and salts thereof for example those providing zinc ions, stannous ions, copper ions, and/or mixtures thereof; bisbiguanides, or phenolics; antibiotics such as augmentin, amoxicillin, tetracycline, doxycycline, minocycline, and metronidazole; and analogs and salts of the above antimicrobial antiplaque agents; anti-fungals such as those for the treatment of candida albicans. If present, these agents generally are present in a safe and effective amount for example from about 0.1% to about 5% by weight of the compositions of the present invention.

Antiinflammatory Agents

Anti-inflammatory agents may also be present in the oral compositions of the present invention. Such agents may include, but are not limited to, non-steroidal anti-inflammatory agents such as aspirin, ketorolac, flurbiprofen sodium, ibuprofen, acetaminophen, diflunisal, fenoprofen calcium, naproxen, indomethacin, ketoprofen, tolmetin sodium, piroxicam and meclofenamic acid, COX-2 inhibitors such as valdecoxib, celecoxib and rofecoxib, and mixtures thereof. If present, the anti-inflammatory agents generally comprise from about 0.001% to about 5% by weight of the compositions of the present invention. Ketorolac is described in U.S. Pat. No. 5,626,838, issued May 6, 1997.

H-2 Antagonists

The present invention may also include a safe and effective amount of a selective H-2 antagonist. Selective H-2 antagonists include compounds which are disclosed in U.S. Pat. Nos. 5,294,433 and 5,364,616 Singer et al., issued Mar. 15, 1994 and Nov. 15, 1994 respectively and assigned to Procter & Gamble, wherein the selective H-2 antagonist is selected from the group consisting of cimetidine, etintidine, ranitidine, ICIA-5165, tiotidine, ORF-17578, lupitidine, donetidine, famotidine, roxatidine, pifatidine, lamtidine, BL-6548, BMY-25271, zaltidine, nizatidine, mifentidine, BMY-25368 (SKF-94482), BL-6341A, ICI-162846, ramixotidine, Wy45727, SR-58042, BMY-25405, loxtidine, DA-4634, bisfentidine, sufotidine, ebrotidine, HE-30-256, D-16637, FRG-8813, FRG-8701, impromidine, L-643728, and HB408. Particularly preferred is cimetidine (SKF-92334), N-cyano-N′-methyl-N″-(2-(((5-methyl-1H-imidazol-4-yl)methyl)thio)ethyl)guanidine:

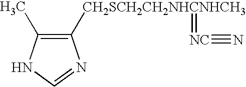

Cimetidine is also disclosed in the Merck Index, 11th edition (1989), p. 354 (entry no. 2279), and Physicians' Desk Reference, 46th edition (1992), p. 2228. Related preferred H-2 antagonists include burimamide and metiamide.

Nutrients

Nutrients may improve the condition of the oral cavity and can be included in the oral care compositions of the present invention. Nutrients include minerals, vitamins, oral nutritional supplements, enteral nutritional supplements, and mixtures thereof.

Minerals that can be included with the compositions of the present invention include calcium, phosphorus, fluoride, zinc, manganese, potassium and mixtures thereof. These minerals are disclosed in Drug Facts and Comparisons (loose leaf drug information service), Wolters Kluer Company, St. Louis, Mo., © 1997, pp10-17.

Vitamins can be included with minerals or used separately. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Such vitamins are disclosed in Drug Facts and Comparisons (loose leaf drug information service), Wolters Kluer Company, St. Louis, Mo., ©1997, pp. 3-10.

Oral nutritional supplements include amino acids, lipotropics, fish oil, and mixtures thereof, as disclosed in Drug Facts and Comparisons (loose leaf drug information service), Wolters Kluer Company, St. Louis, Mo., ©1997, pp. 54-54e. Amino acids include, but, are not limited to L-Tryptophan, L-Lysine, Methionine, Threonine, Levocarnitine or L-carnitine and mixtures thereof. Lipotropics include, but, are not limited to choline, inositol, betaine, linoleic acid, linolenic acid, and mixtures thereof. Fish oil contains large amounts of Omega-3 (N-3) Polyunsaturated fatty acids, eicosapentaenoic acid and docosahexaenoic acid.

Antioxidants that may be included in the oral care composition or substance of the present invention include, but are not limited to Vitamin E, ascorbic acid, Uric acid, carotenoids, Vitamin A, flavonoids and polyphenols, herbal antioxidants, melatonin, aminoindoles, lipoic acids and mixtures thereof.

Enteral nutritional supplements include, but, are not limited to protein products, glucose polymers, corn oil, safflower oil, medium chain triglycerides as disclosed in Drug Facts and Comparisons (loose leaf drug information service), Wolters Kluer Company, St. Louis, Mo., ©1997, pp. 55-57.

Desensitizing Agents and Anesthetic Agents

Anti-pain or desensitizing agents and anesthetic agents can also be present in the oral care compositions or substances of the present invention. Such agents may include, but are not limited to, strontium chloride, potassium nitrate, natural herbs such as gall nut, Asarum, Cubebin, Galanga, scutellaria, Liangmianzhen, Baizhi, etc. Anesthetic agents include lidocaine, benzocaine, etc.

Pharmaceutical Active Agent

The pharmaceutical active agent suitable for use herein is selected from the group consisting of sedatives, hypnotics, antibiotics, antitussives, antihistamines, non-sedating antihistamines, decongestants, expectorants, mucolytics, antidiarrheals, analgesics-antipyretics, proton pump inhibitors, general nonselective CNS stimulants, drugs that selectively modify CNS function, antiparkinsonism drugs, narcotic-analgesics, psychopharmacological drugs, laxatives, dimenhydrinates, and mixtures thereof. Preferred pharmaceutical actives suitable for use as an active ingredient herein include antitussives, antihistamines, non-sedating antihistamines, decongestants, expectorants, mucolytics, analgesics-antipyretics, anti-inflammatory agents, antidiarrheals, and mixtures thereof. The pharmaceutical active agent is included in the oral care compositions at concentrations ranging from about 0.01% to about 50%, preferably from about 0.1% to about 20%, more preferably from about 0.5% to about 10%, even more preferably from about 1% to about 9%, by weight of the dry film composition.

Specific nonlimiting examples of sedatives and hypnotics suitable for use as a pharmaceutical active ingredient herein include those sedatives and/or hypnotics which can provide for a therapeutic benefit in the treatment of sleep disorders. Suitable specific sedatives and hypnotics include doxylamines including doxylamine succinate, melatonins, benzodiazepines including midazolam and triazolam, piperazines, clonidines, nitroglycerins, imidazopyridines, pyrazolopyrimidines, pharmaceutical salts thereof, and mixtures thereof. Doxylamines are most preferred. An example of a commercially available preferred doxylamine pharmaceutical active is doxylamine succinate commercially available from Ganes Chemicals Ltd. located in Pennsville, N.J., USA.

Specific nonlimiting examples of antibiotics suitable for use as a pharmaceutical active ingredient herein include augmentin, amoxicillin, tetracycline, doxycycline, minocycline, metronidazole, and mixtures thereof.

Specific nonlimiting examples of antitussives suitable for use as a pharmaceutical active ingredient herein include those antitussive compounds which are especially effective in treating symptoms of the common cold such as fits of coughing. Suitable specific antitussives include codeine, dextromethorphan, dextrorphan, hydrocodone, noscapine, oxycodone, pentoxyverine, and mixtures thereof. If the drug delivery systems of the present invention comprise an antitussive pharmaceutical active ingredient, dextromethorphan is the most preferred antitussive. As used herein, “dextromethorphan” means racemethorphan, (±)-3-Methoxy-17-methylmorphinan, dl-cis-1,3,4,9,10,10a-hexahydro-6-methoxy-11-methyl-2H-10,4a-iminoethanophenanthrene, and pharmaceutical salts thereof including dextromethorphan hydrobromide. Dextromethorphan and its pharmaceutically-acceptable salts are more fully described in U.S. Pat. No. 5,196,436, issued to Smith on Mar. 23, 1993, which description is incorporated by reference herein.

Specific nonlimiting examples of antihistamines suitable for use as a pharmaceutical active ingredient herein include acrivastine, azatadine including azatadine maleate, brompheniramine, brompheniramine maleate, dexbropheniramine, chlorpheniramine, chlorpheniramine maleate, dexchlorpheniramine maleate, carbinoxamine maleate, clemastine including clemastine fumarate, cyproheptadine, dexbrompheniramine, dimenhydrinate, diphenhydramine, diphenhydramine hydrochloride, diphenhydramine citrate, diphenylpyraline hydrochloride, hydroxyzine, meclizine, pheninamine, phenyltoloxamine, promethazine, promethazine hydrochloride, pyrilamine, pyrilamine maleate, tripelennamine, tripelennamine citrate, triprolidine, triprolidine hydrochloride, and mixtures thereof.

Specific nonlimiting examples of non-sedating antihistamines suitable for use as a pharmaceutical active ingredient herein include astemizole, cetirizine, ebastine, fexofenadine, loratidine, terfenadine, and mixtures thereof.

Specific nonlimiting examples of decongestants suitable for use as a pharmaceutical active ingredient herein include phenylpropanolamine, pseudoephedrine, pseudoephedrine hydrochloride, pseudoephedrine sulfate, ephedrine, phenylephrine, phenylephrine hydrochloride, oxymetazoline, and mixtures thereof.

Specific nonlimiting examples of expectorants suitable for use as a pharmaceutical active ingredient herein include ammonium chloride, guafenesin, ipecac fluid extract, potassium iodide, terpin hydrate, and mixtures thereof.

Specific nonlimiting examples of mucolytics suitable for use as a pharmaceutical active ingredient herein include acetylcycsteine, ambroxol, bromhexine, and mixtures thereof.

Specific nonlimiting examples of antidiarrheals suitable for use as a pharmaceutical active ingredient herein include loperamide and the like.

Specific nonlimiting examples of analgesics-antipyretics suitable for use as a pharmaceutical active ingredient herein include sodium salicylate, salicylamide, indomethacin, phenylbutazone, phenacetin, and mixtures thereof.

Specific nonlimiting examples of proton pump inhibitors suitable for use as a pharmaceutical active ingredient herein include omerprazole, omerprazole magnesium, lansoprazole, and mixtures thereof.

Specific nonlimiting examples of general nonselective CNS stimulants suitable for use as a pharmaceutical active ingredient herein include caffeine, nicotine, strychnine, picrotoxin, pentylenetetrazol, and mixtures thereof.

Specific nonlimiting examples of suitable drugs that selectively modify CNS function include phenyhydantoin, phenobarbital, primidone, carbamazepine, ethosuximide, methsuximide, phensuximide, trimethadione, diazepam, phenacemide, pheneturide, acetazolamide, sulthiame bromide, gabapentin, phenyloin, and mixtures thereof.

Specific nonlimiting examples of antiparkinsonism drugs suitable for use as a pharmaceutical active ingredient herein include levodopa, amantadine, and mixtures thereof.

Specific nonlimiting examples of narcotic-analgesics suitable for use as a pharmaceutical active ingredient herein include morphine, heroin, hydromorphone, metopon, oxymorphone, levorphanol, codeine, hydrocodone, oxycodone, nalorphine, naloxone, naltrexone, and mixtures thereof.

Specific nonlimiting examples of psychopharmacological drugs suitable for use as a pharmaceutical active ingredient herein include chlorpromazine, methotrimeprazine, haloperidol, clozapine, reserpine, imipramine, tranylcypromine, pheneizine, lithium, and mixtures thereof.

### Other Optional Ingredients

Surfactants

The present composition optionally comprises a safe and effective amount of a surfactant, in another embodiment comprises from about 0.001% to about 20%, in another embodiment from about 0.05% to about 6%, and in even another embodiment from about 0.1% to about 3% by weight of the composition of surfactant. On the other hand, edible film compositions that have no or low levels of surfactant exhibit improved shelf-life of the flavor components, during short term (1-7 days) and long term storage (8-90 days). This advantage is due in part, to an increase in the edible films resistance to environmental moisture. Therefore, in another embodiment the present compositions have less than about 1%, in another embodiment have less than about 0.5%, by weight surfactant, and in yet another embodiment are essentially free of surfactants.

Suitable surfactants are those which are reasonably stable and include nonionic, anionic, amphoteric, cationic, zwitterionic, synthetic detergents, and mixtures thereof. Many suitable nonionic and amphoteric surfactants are disclosed by U.S. Pat. No. 3,988,433 to Benedict; U.S. Pat. No. 4,051,234, issued Sep. 27, 1977, and many suitable nonionic surfactants are disclosed by Agricola et al., U.S. Pat. No. 3,959,458, issued May 25, 1976.

Sweetening Agents, Coolants, Salivating Agents, Warming Agents

The present compositions may optionally comprise sweetening agents including sucralose, sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. A composition preferably contains from about 0.1% to about 10% of these agents, in another embodiment from about 0.1% to about 1%, by weight of the composition.

Coolants, salivating agents, warming agents, and numbing agents can be used as optional ingredients in compositions of the present invention. These agents are present in the compositions at a level of from about 0.001% to about 10%, in another embodiment from about 0.1% to about 1%, by weight of the composition.

The coolant can be any of a wide variety of materials. Included among such materials are carboxamides, menthol, ketals, diols, and mixtures thereof. Preferred coolants in the present compositions are the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979.

Preferred salivating agents of the present invention include Jambu® manufactured by Takasago. Preferred warming agents include capsicum and nicotinate esters, such as benzyl nicotinate. Preferred numbing agents include benzocaine, lidocaine, clove bud oil, and ethanol.

### Method of Making Film Compositions

The film compositions utilized in accordance with the invention are formed by processes conventional in the arts, e.g. the paper-making and/or film making industries. Generally the separate components of the film are blended in a mixing tank until a homogeneous mixture is achieved. Thereafter, the films can be cast to an acceptable thickness, on an appropriate substrate. Examples of such substrates include Mylar, continuous moving stainless steel belt (eventually entering a dryer section), release paper and the like. The webs are then dried, e.g. in a forced-air oven. The temperature of the drying air and length of drying time depend on the nature of the solvent utilized as is recognized in the art. Most of the films contemplated herein, however, are dried at a temperature between about 25° C. and 140° C., in another embodiment from about 60° and 90° C. for a duration of about 20 minutes to about 60 minutes, in another embodiment from about 30 to about 40 minutes. After exiting from the dryer section of the casting belt, the film can be wound on a spool for storage under sanitary conditions. The film can be slit into two inch rolls for further cutting to form 1 inch by 2 inch (or other desired dimensions) and then stacked and subsequently individually packaged.

Another conventional film-making process known in the art is extrusion. This method is possible with films wherein the film forming ingredient comprises a variety of materials, for example, a modified food starch, hydroxypropylcellulose or other extrudable polymer. The mechanical particulars of the extrusion process, e.g. the particular equipment utilized, the extruding force, the shape and temperature of the orifice are considered to be within the skill of the art and can be varied in a known manner to achieve the physical characteristics of the films described herein.

The films herein are generally between about 1 and about 10 mils (about 0.025 mm to about 0.25 mm), in another embodiment are from about 1.2 to about 2.5 mils (about 0.03 mm to about 0.063 mm) thick. A convenient width for such films is about 0.75 to about 1 inch, although the width of the film is not particularly critical to the practice of the invention. The film can be produced in any length. However, in view of the fact that the novel dosage forms produced in accordance with the invention are suited to high speed manufacture, the films should be prepared in large quantity, e.g. 15,000 feet or more which can be stored, e.g. on cores or spools.

The film forming agent can be added with the other ingredients to form a homogeneous mixture.

### Composition Use

Generally, the subject places the film in the oral cavity where the film dissolves completely either rapidly or over 1-8 hours. The frequency of use by the subject is preferably from about once per week to about ten times per day, in another embodiment from about thrice per week to about five times per day, in even another embodiment from about once per day to about twice per day. The period of such treatment typically ranges from about one day to a lifetime. For particular oral care diseases or conditions the duration of treatment depends on the severity of the oral disease or condition being treated, the particular delivery form utilized and the patient's response to treatment. In one embodiment the duration of treatment is from about 3 weeks to about 3 months, but may be shorter or longer depending on the severity of the condition being treated, the particular delivery form utilized and the patient's response to treatment.

The compositions of this invention are useful for both human and other animals (e.g. pets, zoo, or domestic animals).

### EXAMPLES

The following non-limiting examples further describe preferred embodiments within the scope of the present invention. Many variations of these examples are possible without departing from the scope of the invention.

### Example I

Edible film compositions are described below:

| | (% By Wt. Wet) | | | | |
|---|---|---|---|---|---|
| Ingredient | Example 1 | Example 2 | Example 3 | Example 4 | Example 5 |
| Water | 70.76% | 71.15% | 60.21% | 76.30% | 70.35% |
| HPMC | 3.00% | 6.00% | 3.00% | 5.00% | 5.00% |
| Methocel | | | | | |
| K3¹ | | | | | |
| HPMC | — | — | 3.00% | 1.00% | — |
| Methocel | | | | | |
| E50¹ | | | | | |
| HPMC | 2.00% | 2.00% | 2.00% | — | 3.00% |
| Methocel | | | | | |
| K100¹ | | | | | |
| HPMC | 0.50% | 0.50% | — | 0.90% | — |
| Methocel | | | | | |
| K4M¹ | | | | | |
| HPMC | — | — | — | — | 1.00% |
| Methocel | | | | | |
| E4M¹ | | | | | |
| Indigestible | 6.50% | 6.30% | 9.00% | 5.50% | 7.00% |
| Dextrin | | | | | |
| Acesulfame | 0.50% | 0.80% | 0.90% | 1.80% | 0.90% |
| Potassium | | | | | |
| Sucralose | — | 0.50% | 0.45% | — | 0.80% |
| Dextrin | 1.00% | 3.00% | 5.00% | 1.00% | 5.00% |
| Aspartame | 0.90% | — | 0.50% | — | — |
| Citric Acid | 0.50% | 1.00% | 1.10% | 1.00% | 1.00% |
| Flavor Oil | 7.00% | 5.00% | 8.00% | 4.00% | 3.00% |
| Canola Oil | 2.00% | 1.00% | 2.00% | — | — |
| Gum Arabic | 2.00% | 1.00% | 2.00% | 2.00% | 1.45% |
| Color | 1.00% | 0.75% | 0.50% | 0.50% | 0.50% |
| Sorbitol | 2.34% | 1.00% | 2.34% | 1.00% | 1.00% |
| Total | 100.00% | 100.00% | 100.00% | 100.00% | 100.00% |
| | (% By Wt. Wet) | | | | |
| | | | | | Example |
| Ingredient | Example 6 | Example 7 | Example 8 | Example 9 | 10 |
| Water | 70.60% | 72.35% | 71.16% | 60.31% | 76.10% |
| HPMC | 4.00% | 8.00% | 3.00% | 3.00% | 5.00% |
| Methocel | | | | | |
| K3¹ | | | | | |
| HPMC | 5.00% | — | — | 3.00% | 1.00% |
| Methocel | | | | | |
| E50¹ | | | | | |
| HPMC | — | 1.00% | 2.00% | 2.00% | — |
| Methocel | | | | | |
| K100¹ | | | | | |
| HPMC | — | 0.90% | 0.50% | — | 0.90% |
| Methocel | | | | | |
| K4M¹ | | | | | |
| HPMC | — | — | — | — | — |
| Methocel | | | | | |
| E4M¹ | | | | | |
| Indigestible | 10.50% | 1.00% | 6.50% | 9.00% | 5.50% |
| Dextrin | | | | | |
| Acesulfame | — | 0.90% | 0.50% | 0.70% | 1.50% |
| Potassium | | | | | |
| Sucralose | — | 0.90% | — | 0.45% | — |
| Dextrin | 0.90% | 1.00% | 1.00% | 5.00% | 1.00% |
| Aspartame | 1.80% | — | 0.90% | 0.74% | — |
| Citric Acid | 1.00% | 1.00% | 0.10% | 1.00% | 1.00% |
| Flavor Oil | 3.00% | 7.50% | 2.75% | 3.25% | 2.00% |
| Menthol | — | — | 4.25 | 4.75% | 2.50% |
| Canola Oil | — | — | 2.00% | 2.00% | — |
| Gum Arabic | 1.70% | 2.00% | 2.00% | 2.00% | 2.00% |
| Color | 0.50% | 1.25% | 1.00% | 0.50% | 0.50% |
| Sorbitol | 1.00% | 2.20% | 2.34% | 2.30% | 1.00% |
| Total | 100.00% | 100.00% | 100.00% | 100.00% | 100.00% |

¹Available from Dow Chemical Company

To produce the film formulations of examples 1-3 and 8-9, add the film forming agents (Methocel variants) to a mixture containing canola oil, flavoring agent, menthol if desired, and sorbitol. Then agitate this mixture until the particles of Methocel powder are homogenously dispersed. Water, at a temperature of approximately 75° C. is then added and agitation is continued for at least 30 minutes. Then add the remaining ingredients, such as color, sweeteners, and the indigestible dextrin, to the solution and mix under agitation for at least 10 minutes. Pour the casting solution onto a glass plate and drawn down to form a thin monlayer film. Then dry the film for ten minutes at 70° C. Next, remove the film from the glass plate and cut into the desired dimensions.

To produce the film formulations of examples 4-5 and 10, heat the water to greater than 180° F. Then add the Methocel variants to the hot water and mix at 180° F. for at least 10 minutes. Follow by adding the remaining ingredients to the hot mixture, such as color, sweeteners, and indigestible dextrin. The mixture is then mixed for at least 5 minutes. Cool the casting solution to 25° C. and pour onto a glass plate and drawn down to form a thin monolayer film. Next dry the film for fifteen minutes at 70° C. Next, remove the film from the glass plate and cut into the desired dimensions.

For Examples 6 and 7 thoroughly mix the Methocel variants with dextrin and gum Arabic. Then add this dry mixture to water under high agitation. Continue the agitation for at least 30 minutes. The remaining ingredients, such as color, sweeteners, and the indigestible dextrin, are then added to the solution and mixed under agitation for at least 10 minutes. Next pour the casting solution onto a glass plate and drawn down to form a thin monolayer film. Then dry the film for fifteen minutes at 70° C. Next, remove the film from the glass plate and cut into the desired dimensions.

While particular embodiments of the present invention have been described, it will be obvious to those skilled in the art that various changes and modifications of the present invention can be made without departing from the spirit and scope of the invention. It is intended to cover, in the appended claims, all such modifications that are within the scope of this invention.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An edible film composition comprising:
a. a safe and effective amount of a cellulose based film forming agent comprising a mixture of at least one low viscosity cellulose based film forming agent and at least one high viscosity cellulose based film forming agent;
b. a safe and effective amount of a plasticizing agent; and
c. a safe and effective amount of a flavoring agent;
wherein the film composition rapidly dissolves in the oral cavity.

2. The composition of claim 1 wherein the low viscosity film forming agent has a viscosity of about 1 to about 40 mPa.s.

3. The composition of claim 2 wherein the low viscosity film forming agent has a viscosity of about 2 to about 20 mPa.s.

4. The composition of claim 2 wherein the high viscosity film forming agent has a viscosity of about 50 to about 10,000 mPa.s.

5. The composition of claim 4 wherein the high viscosity film forming agent has a viscosity of about 100 to about 5,000 mPa.s.

6. The composition of claim 4 wherein at least one of the film forming agents is HPMC.

7. The composition of claim 6 wherein at least one of the film forming agents is HPMC with a 19-24% methoxy group substitution and a 7-12% hydroxyproproxyl group substitution.

8. The composition of claim 1 wherein the total level of film forming agent is from about 2% to about 30% by weight of the wet film composition.

9. The composition of claim 8 wherein the total level of film forming agent is from about 4% to about 7% by weight of the wet film composition.

10. The composition of claim 8 wherein the film forming agent is selected from the group consisting of hydroxypropyl cellulose, hydroxypropyl methyl cellulose, and mixtures thereof.

11. The composition of claim 1 wherein the composition further comprises a safe and effective amount of a vegetable oil.

12. An edible film composition comprising:
a. a safe and effective amount of a cellulose based film forming agent comprising a mixture of at least one low viscosity cellulose based film forming agent and at least one high viscosity cellulose based film forming agent;
b. a safe and effective amount of a plasticizing agent; and
c. a safe and effective amount of a flavoring agent;
wherein the film composition rapidly dissolves in the oral cavity and wherein the composition is essentially free of surfactant.

13. A method of increasing the film strength of an edible film composition by incorporating into the film composition, a safe and effective amount of a cellulose based film forming agent comprising a mixture of at least one low viscosity cellulose based film forming agent and at least one high viscosity cellulose based film forming agent.

14. A method of treating an oral condition by administering a safe and effective amount of the composition of claim 1 to the oral cavity of a subject in need thereof.
